(19) United States
(12) Patent Application Publication
(10) Pub. No.: US 2008/0086069 A1
(43) Pub. Date: Apr. 10, 2008

(54) Wrist support brace

(76) Inventor: Robert Busuttil, Pomona, CA (US)

Correspondence Address: Lewis M. Brande, 5976 Falling Tree Lane, Alta Loma, CA, 91737-2816, US

(21) Appl. No.: 11/728,340

(22) Filed: Mar. 24, 2007

Related U.S. Application Data
(60) Provisional application No. 60/851,094, filed on Oct. 10, 2006.

(51) Int. Cl. A61F 5/01

(52) U.S. Cl. 602/21

(57) ABSTRACT

This invention describes a novel wrist support brace for patients with carpal tunnel syndrom or injured wrists that utilizes an adjustable strap in conjunction with a hard support brace positioned around the forearm. The brace will physically contain the injured wrist in a stable position, thereby minimizing the potential of further injury. The wrist brace will allow some movement of the wrist and permit a user to continue working while the wrist heals in a normal position.

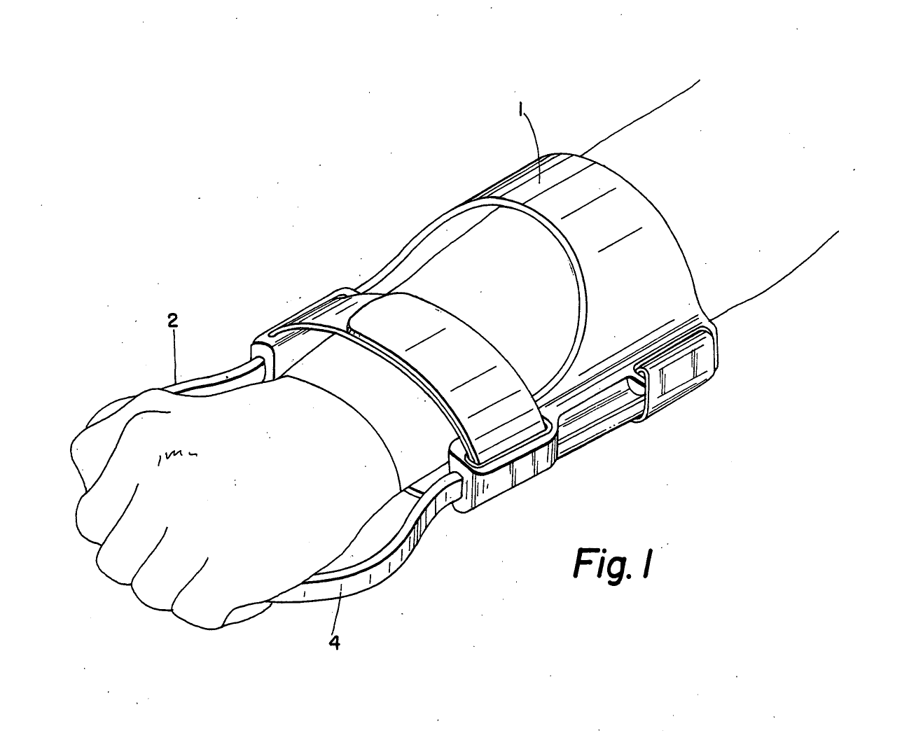

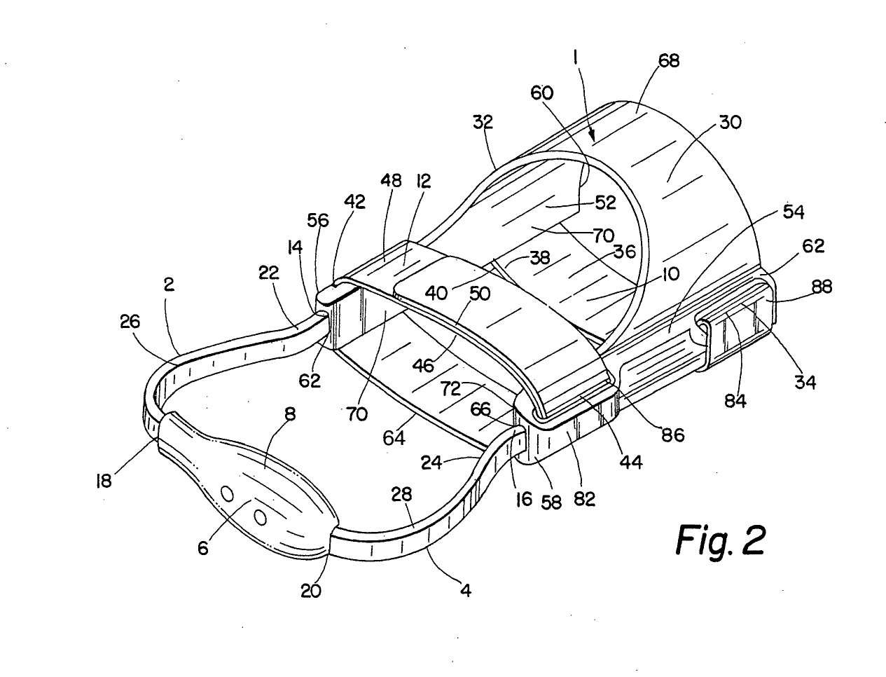

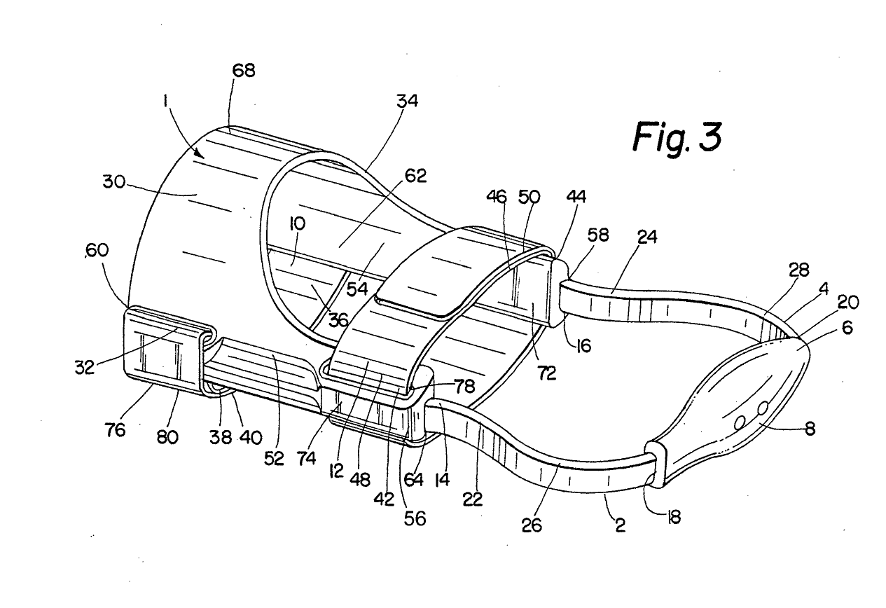

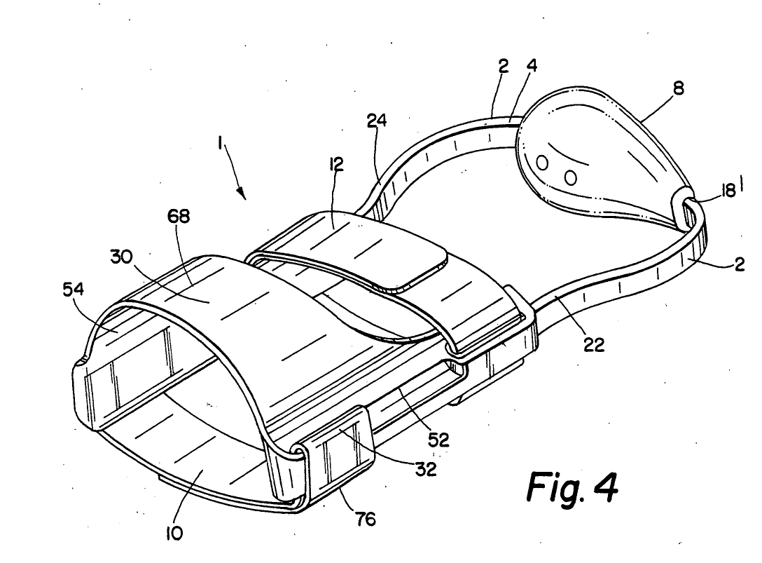

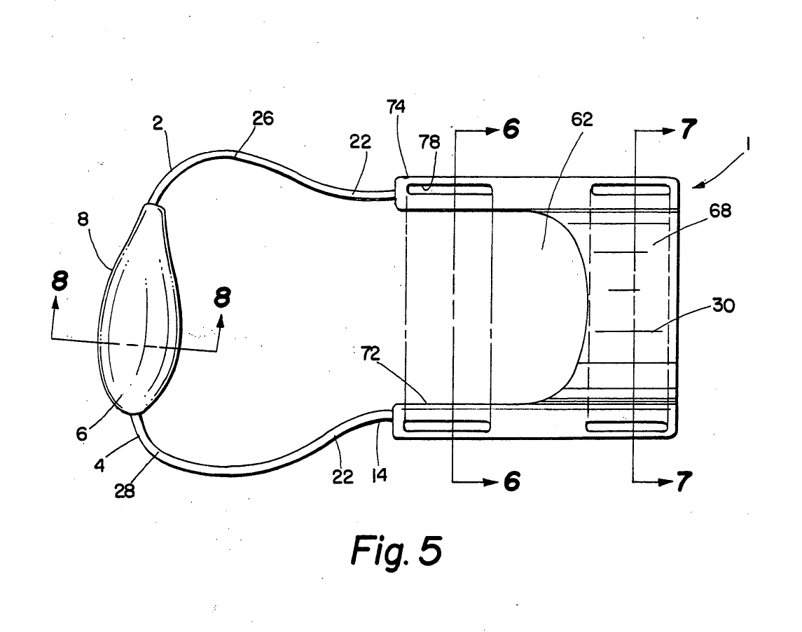

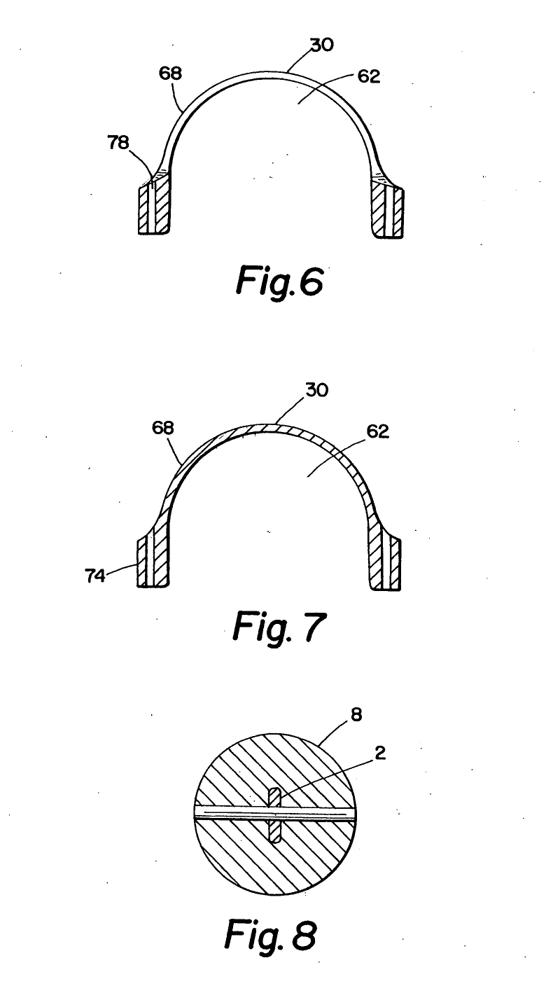

This patent claims priority of provisional patent No. 60/851094, filed Oct. 10, 2006.

### 1. BACKGROUND OF THE INVENTION

This invention is used in combination with and comprises an improvement to U.S. Pat. No. 7,033,332 and relates to the field of wrist supports. More specifically the invention relates to an device in the field of orthotics. The device is used to assist a person who suffers from forms of strokes, paralysis, or other forms of musculature or nerve disease or damage that require manual straightening of the wrist to restore the appendage to normal everyday use.

### 2. DESCRIPTION OF THE PRIOR ART.

Various types of races and appliances for therapeutic use in treating bone or muscle disorders are described in the prior art. Several patents exist to assist patients in restoring a hand that is contracted to normal condition.

A typical device is described by U.S. Pat. No. 5,437,620 by Shelly which discloses a “Wrist Splint”. This device bladders that assist in supporting the hand. The wrist is supported by rigid nonpliable platform. Straps are used that wrap around the platform and over the wrist, applying pressure to straighten the wrist during the contractions.

Another device is U.S. Pat. No. 5,601,597 by Arrowood et al., which discloses “Combination Radial Artery Occluder and Wrist Splint”. This device is specifically used to immobilize a wrist in order to prevent further damage or injury to an artery. The device is made specifically to prevent flow through the radial artery by applying pressure and allowing blood flow through the ulnar artery. Although the device does comprise a rigid member, the rigid member is not specifically needed to straighten the wrist, but only to maintain the wrist in an immobile state.

U.S. Pat. No. 5,772,620 by Szlema et al. Discloses a “Hand and Wrist Joint Orthosis”. This device was created to immobilize an injured hand. The wrist and the fingers are maintained in a specific orientation to each other to guarantee good recovery from an injury. The device is not designed to be used for wrist contractures, in where the wrist must be forcibly dislocated.

U.S. Pat. Nos. 6,106,492, 6,723,061, and 6,045,517 are inventions that are specifically related to treatment of carpal tunnel injuries. These devices immobilize the wrist and are designed to prevent unwanted movement of the wrist, which will cause further injury to the carpal nerve system in the wrist. The devices are distinguished from the present invention in that they are not designed to apply the force necessary to forcibly straighten the wrist that has been stricken with a contracture.

U.S. Pat. No. 6,547,752 discloses an “Orthotic Device for Treating Contractures of Either Hand”. This device uses a soft padded envelope or mitt that is mated with a splint. The device has padded attachment straps that are located at different regions of the mitt. This device uses a mitt, which is made from a manually bendable material such as metal encased in a soft plastic padding. It should be noted that there is a great difference to the present invention in that the present invention is not made from bendable flat sheet metal, which is needed for Patent Publication Number US 2002/0082538 A1 to be built. Additionally the method by which the straps are used is different from the present invention, in that the straps to U.S. Pat. No. 6,547,752 wrap over the wrist making it very difficult for a sole person, without assistance, to apply enough pulling force onto the straps to cause the constricted appendage to be forcibly straightened.

U.S. Pat. No. 7,033,332 by Busuttil, discloses a “Wrist Support Brace for Alleviating Contractures”, and is the parent invention from which the improvements are applied

### 3. SUMMARY OF THE INVENTION.

In accordance with the present invention, there is provided a new and novel orthotic device that has been designed to assist a person in alleviating contractures and mild pain, i.e carpal tunnel syndrom of a person's wrist. This particular device maybe used for either the left or right hand. This device has been made by bending a metallic bar that may be made from metals such as aluminum, titanium, or steel. The metal is stiff enough to prevent a user from bending the device out of shape. The device comprises two essentially parallel bars, one on each side of the arm that extend towards the arm. The two bars follow a 90th percentile (90%) persons wrist/hand shape and are joined at the hand. The general shape would be considered a deep “C” shape, where the two bars are joined by a elongated hand support region. Foam is wrapped around the hand area to provide the user with a easily gripped surface. At least two velcro straps are included with the orthotic device, and wrap around the wrist and the forearm.

A first improvement on the previous invention is the addition of a forearm support, where the forearm support is attached to the two parallel bars on each side of the forearm. A cradle may be integrally attached to the two forearm supports which provides positional support for the forearm, and allows the forearm to rest inside the cradle. A further improvement is that the cradle provides additional stability and stiffness to the two parallel bars.

A second improvement on the previous invention is that the forearm support is adapted to attach the velcro straps which improves the visual appeal of the apparatus, and prevents premature wear of the velcro straps.

A third improvement on the previous invention is to make the grip pad more ergonomic in design. This would allow a different material used, such as rubber, plastic, or composite, which is more durable than foam. The ergonomic hand grip may also be made from a solid material with an elastomeric covering such as rubber, silicone or foam on the outside, where the solid inner material would provide a hard gripping inner support while the elastomeric covering provides gripping comfort for the user.

It is the primary object of the present invention to provide an orthotic device that can treat and stabilize carpal tunnel injuries to the and also treat contractures of the wrist or hand.

It is a further object of the invention to provide an orthotic device that would allow a patient to easily apply a force perpendicular to the contracture without the assistance of another person.

A still further object of the invention is to provide an orthotic device that is easily fabricated and may be used on either wrist or hand.

Yet another object of the invention is to provide an orthotic device that has a designed shape that provides the smallest profile form, thus preventing serious interference with a patients normal use of his wrist or hand.

These and other objects, features and advantages of the present invention will be come apparent to those skilled the art from the following detailed description and attached drawings, upon which, by way of example, only a preferred and other important embodiments of the invention are described and illustrated.

### 4. BRIEF DESCRIPTION OF THE DRAWINGS

1. Figure one (1) shows a left perspective view of the wrist support brace with a users arm in place;

2. FIG. (2) shows a left perspective view of the wrist support brace

3. Figure three (3) shows a right perspective view of the wrist support brace;

4. Figure four (4) shows aright rear perspective view of the wrist support brace; and

5. Figure Five (5) shows a plan view of the wrist support brace.

### 5. DETAILED DESCRIPTION

With respect to figures one through five (1-5), what is shown is a wrist support brace (1). The wrist support brace (1) is shown having an ulnar support (2), and a radial support (4). The ulnar support (2) and the radial support (4) may be positioned in a parallel relationship as shown in FIG. four, or in a diverging, non-parallel relationship. A first end (14) of the ulnar support (2), and a first end (16) of the radial support (4) are spaced further apart than a second end (18) of the ulnar support (2) and a second end (20) of the radial support (4). The second end (20) or the radial support (4) and the second end (18) ulnar support (2) are joined by a grip support (6) with a grip pad (8).

With respect to FIG. four (4), the wrist support brace (I) is shown in plan view. The ulnar support (2) is shown having a ulnar support kick point (22), and the radial support (4) is shown having a radial support kick point {24). The ulnar support (2) is bent about the ulnar support kick point (22), and the radial support (4) is bent about the radial support kick point (24). A first bent portion (26) of the ulnar support (2) and a first bent portion (28) of the radial support (4) are positioned in an approximate parallel relationship, wherein the grip support (6) is shown attached the first bent portion (26) of the ulnar support (2) and the first bent portion (28) of the radial support (4) in nearly a right angle orientation. In order to maximize the utility of the wrist support brace (1), the ulnar support (2), the radial support (4), and the wrist support (6) are shown lying in a planar relationship.

For maximum comfort, the first bent portion (26) of the ulnar support (2) and the first bent portion (28) of the radial support (4) are shown having a space between a users closed fist and the first bent portion (26) of the ulnar support (2) and the first bent portion (28) of the radial support (4).

A forearm cradle (30) is defined as having an ulnar bracket (52) and a radial bracket (54). The ulnar and radial brackets (52, 54) each have a first end (56, 58) and a second end (60, 62). The ulnar bracket has a first slot {64) defined therein, where the first slot (64) penetrates through the first end (56) of the ulnar bracket (52) but may not penetrate the second end (60). The radial bracket (54) is similarly made in that the radial bracket has a second slot (66) defined therein, where the second slot (66) penetrates through the first end (58) and may not penetrate through the second end (62). The ulnar support (2) is inserted through the first slot (64) and is fixed in position to prevent any unwanted motion. The radial support (4) is inserted through the second slot (66) and is fixed in position to prevent any unwanted motion. A forearm support (68) is shown attaching the ulnar bracket (52) and the radial bracket (54). The forearm support (68) is shown as semi circular in shape, but may be made in any geometric shape that provides comfort to the user o r the wrist brace (1). The ulnar and radial brackets (52, 54) each have a flat side (70, 72), where the flat side (70) of the ulnar bracket (52) faces the users forearm, and the flat side (72) of the radial bracket (54) also faces the users forearm.

The ulnar bracket (52) has a first boss (74) and a second boss (76) defined thereon, the First boss (74) being located towards the ulnar support kick point (22), and has a first slot (78) defined therein. The second boss (76) is located in proximity to the forearm support (68) and has a second slot (80) defined therein. The radial bracket (54) has a third boss (82) and a fourth boss (84) defined thereon, the first boss (82) being located towards the radial support kick point (24), and has a third slot (86) defined therein. The fourth boss (84) is located in proximity to the forearm support (68) and has a fourth slot (88) defined therein.

The forearm strap (10) has a first ulnar slot (32) and a first radial slot (34) defined therein_The first ulnar slot (32) is looped through the second slot (80) of the second boss (76) of the ulnar bracket (52), and the first radial slot (34) is looped through the fourth slot (86) of the fourth boss (84). The forearm strap (10) is shown being looped below the forearm support (68) and snugs the forearm against the forearm support (68) when drawn tight. A reusable self adhesive portion (36) is typically made from materials similar to Velcro'. The reusable self adhesive portion (36) has a first strap (38) and a second strap (40). The first strap (38) laying on the forearm of a user, the second strap (40) overlapping the first strap (38) and being fastened thereby, fixing the wrist support brace into position.

The adjustable strap (12) is looped through the first slot (78) of the first boss (74) and the third slot (86) of the third boss (82), and acts as a continuous loop, The adjustable strap (12) further has a third strap (48). The third strap (48) wraps over a users wrist, and through the first slot (78) of the first boss (74). A fourth strap (50) is the continuation of the adjustable strap (12). The fourth strap (50), has a second self adhesive portion (46), the second self adhesive portion (46) being constructed from hook and loop materials common within the industry and commonly known as Velcro®. When the user has applied sufficient force to position the wrist the second self adhesive portion (46) will fix the adjustable strap (12) into position, securing the wrist into position.

The grip pad (8) is fixed in position using fasteners (90) that attach to the grip support (6) and prevent any translation or rotational movement. An alternative method of attaching the grip pad (8) is to use adhesive.

In essence, the wrist support brace (1) is operated as follows. A user with a wrist injury will grip the grip pad (8) with his fist. The user will then position the forearm support (68) located on the forearm cradle (30) on the top portion of the user, and wrap the forearm strap (10) under his/her forearm, fixing the wrist support brace (1) in position. The adjustable strap (12) in the improved wrist brace wraps over the wrist. The user applies as much force as necessary by pulling the adjustable strap (4) securing the wrist into the desired wrist stabilizing position. The fourth strap (50), an extension of the adjustable strap (12), is then wrapped over the wrist and adjustable strap (12) where the second self adhesive portion (46) is located. The fourth strap (50) is then attached to the adjustable strap (12) fixing the adjustable strap and thereby immobilizing the wrist to a stable secure position.

Although the foregoing includes a description of the best mode contemplated for carrying out the invention, various modifications are contemplated.

As various modifications could be made in the constructions herein described and illustrated without departing from the scope of the invention, it is intended that all matter contained in the foregoing description or shown in the accompanying drawings shall be interpreted as illustrative rather than limiting.

What is claimed is:

1. A wrist support brace the improvement comprising:
a. an ulnar support, said ulnar support having a first end and an second end, a radial support, said radial support having a first end and a second end, a grip support, said grip support being attached to said second end of said ulnar support and said second end of said radial support, said ulnar support, said radial support and said grip support being defined in a planar relationship, a grip pad, said grip pad being attached to said grip support;
b. in addition, said radial support and said ulnar support are defined in a diverging relationship wherein said second ends of said radial and said ulnar supports being spatially closer than said first ends of said ulnar and said radial supports;
c. a forearm strap, said forearm strap being attached to a first end of said radial support and having self adhesive attach means defined thereon, said forearm strap being attached to said first end of said ulnar support creating a cradle therebetween, a second strap, said second strap wrapping around a patient's forearm and being removably attached to a first strap; and
d. an adjustable strap, said adjustable strap being attached to said radial support and being positionally fixed at an approximate midpoint of said radial support, said adjustable strap having a third strap, said third strap wrapping over a patient's contractured wrist and under said ulnar support, said adjustable strap having a fourth strap with means to removably attach itself to said adjustable strap wherein a patient with a wrist contracture can grip said grip pad, wrap the forearm strap around the patient's forearm contracting the patient's forearm upward, then said adjustable strap is wrapped over the patient's wrist and the patient pulls said adjustable strap forcing the patient's wrist downward thereby into a non-contractured position.

2. The wrist support brace of claim 1, wherein said second end of said ulnar support having a first bent portion, said first bent portion of said ulnar support being bent about an ulnar support kick point, said second end o f said radial support having a first bent portion of said radial support being bent around a radial support kick point said First bent portion of said ulnar support and said first bent portion of said radial support being in an essentially parallel relationship providing clearance to a patient's wrist and fist thereby.

3. A wrist support brace comprising:
a. an ulnar support, a grip support with a grip pad and a radial support, said ulnar support having a first end and a second end, said second end of said ulnar support connected to a first end of said grip support, said radial support having a first end and a second end, said second end of said radial support connected to a second end of said grip support, a first bent portion of said ulnar support being bent about an ulnar support kick point, a first bent portion of said radial support being bent around a radial support kick point in parallel relationship to said first bent portion of said ulnar support;
b. a forearm strap at said first end of said ulnar support and said first end of said radial support, said forearm strap having a first ulnar slot and a first radial slot, s aid first u Mar s lot and s aid second radial slot allowing s aid u Mar support and said radial support to pass through said first ulnar slot and said first radial slot respectively, said forearm strap having a first strap looped around said ulnar support and said radial support forming a forearm cradle, said forearm strap having means to attach itself to a second strap; and
c. An adjustable strap having a second radial slot, said radial support passing through said second radial slot with said second radial slot being positionally fixed at an approximate midpoint of said radial support, said adjustable strap having a third strap which wraps over a patient's contractured wrist and under said ulnar support creating a second ulnar slot, said adjustable strap having a fourth strap with means to attach itself to said adjustable strap wherein a patient with a wrist contracture can grip said grip pad, wrap the forearm strap around the patient's forearm contracting the patient's forearm upward, then said adjustable strap is wrapped over the patient's wrist and the patient pulls said adjustable strap with enough strength to force the patient's wrist downward into a non-contractured position.

4. The wrist support brace according to claim 3 wherein said forearm strap attachment means consists of a hook and loop fastener.

5. The wrist support brace according to claim 3 wherein said adjustable strap attachment means consists of a hook and loop fastener.
